(19) United States
(12) Patent Application Publication
(10) Pub. No.: US 2003/0092635 A1
(43) Pub. Date: May 15, 2003

(54) Galenical preparations of dapsone and related sulphones, and method of therapeutic and preventative treatment of disease

(76) Inventors: A K Gunnar Aberg, Sarasota, FL (US); Alexander Zolotoy, Richmond (CA); Allen I Bain, Vancouver (CA)

Correspondence Address: Kevin S Lemack, Neilds & Lemack, 176 E Main Street, Westboro, MA, 01581, US

(21) Appl. No.: 10/149,108

(22) PCT Filed: Dec. 7, 2000
(86) PCT No.: PCT/US00/33138

(30) Foreign Application Priority Data
Dec. 8, 1999 (US) 60169727

(51) Int. Cl. A61K 31/70; A61K 31/19; A61K 31/135; A61K 31/16

(52) U.S. Cl. 514/23; 514/646; 514/575; 514/629

(57) ABSTRACT

Dapsone and related sulfones are known to have therapeutic activity against leprosy, dermatitis herpetiformis, actinomycotic mycetoma, asthma, malaria, rheumatoid arthritis, Kaposiís sarcoma, pneumocystis carinií (pneumonia), subcorneal pustular dermatosis and cystic acne, in patients in need of such therapy. These sulfones are also known to have therapeutic activity against memory loss in patients in need of such therapy, including patients suffering from Alzheimer's disease and related neurodegenerative disorders. It has now been found that new, modified-release formulations of dapsone and related sulfones may also be used that decrease side effects and increase effectiveness of the drugs. New methods are disclosed utilizing certain formulations of dapsone and related sulfones that improve the therapeutic index of said drugs. Side effects of these drugs are known to those skilled in the art and include, but are not restricted to anorexia, psychosis, agranulocytosis, peripheral neuritis, hemolysis, methemoglobinemia, nausea, vomiting, headache, dizziness, tachycardia, nervousness, insomnia and skin disorders. Modified-release (as defined herein) formulations of dapsone have now been found to avoid some or all of these side effects, and to have more efficacy on potency.

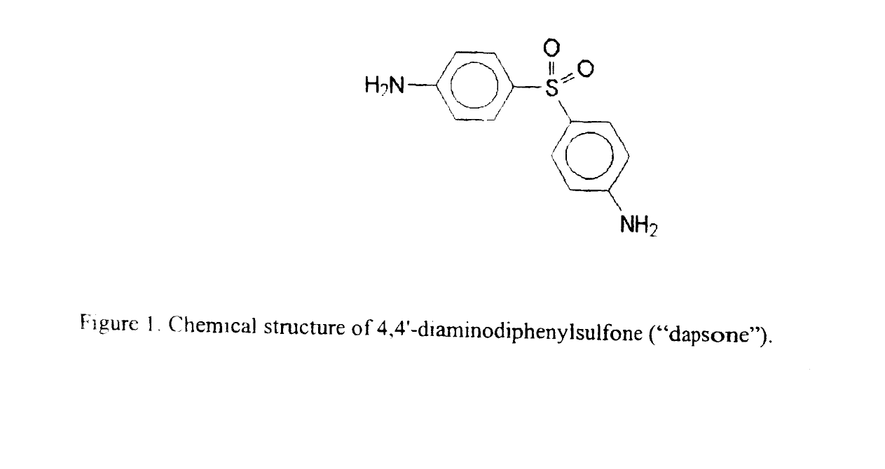

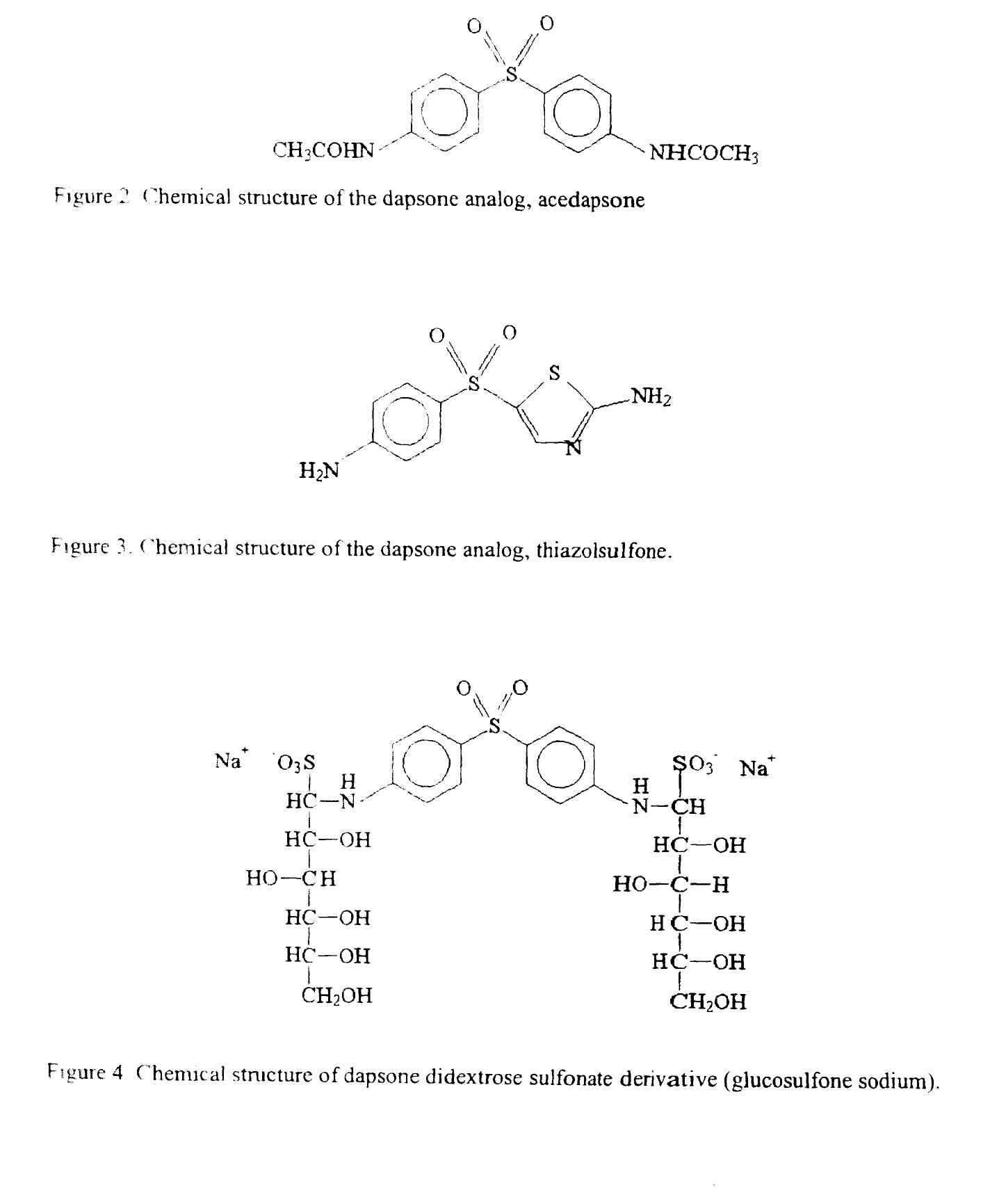

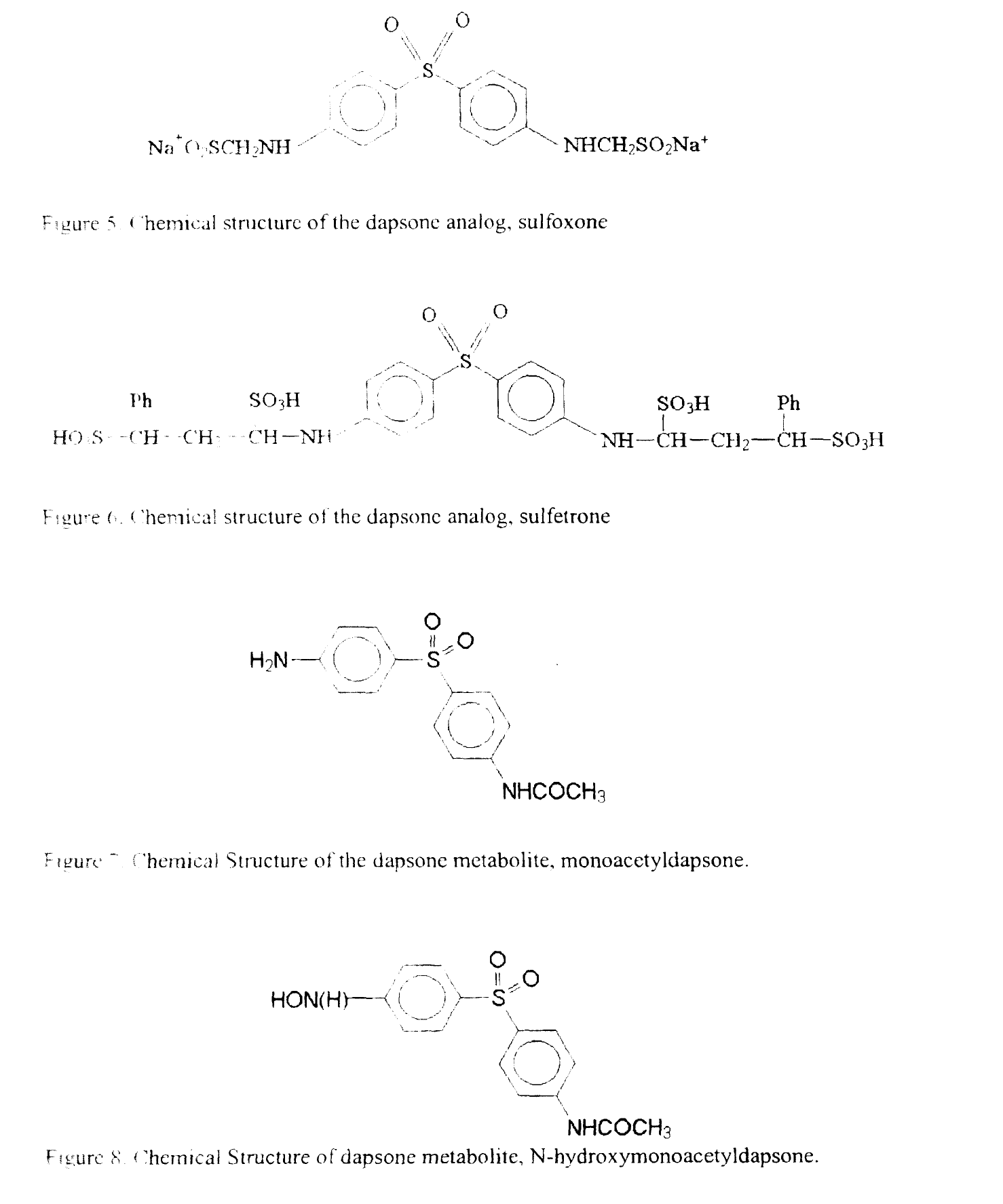

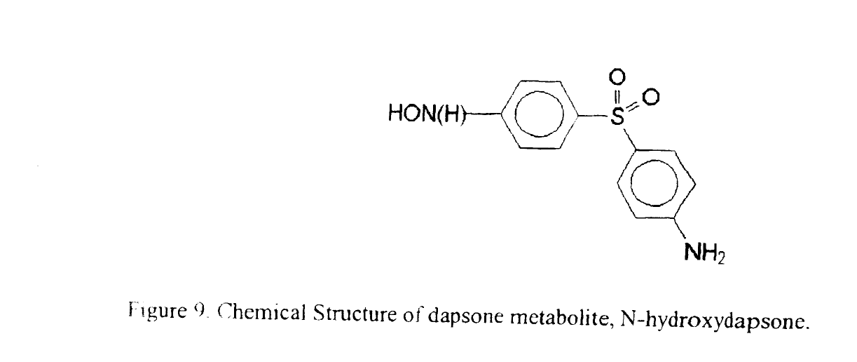

### FIELD OF THE INVENTION

The object of the present invention pertains to a method of treating or preventing certain diseases in a human being while increasing compliance, reducing side effects and improving efficacy of the active therapeutic ingredient(s) within a large therapeutic range. The method comprises the use of modified-release dosage formulations of sulfone compounds including 4,4′-diaminodiphenylsulfone, its didextrose sulfonate derivative(s), their analogs, metabolites, any enantiomers, any diasteriomers, or mixtures thereof and/or therapeutically acceptable salts thereof.

### BACKGROUND OF INVENTION

Dapsone is an active substance that is known in the treatment of various infectious diseases and inflammatory conditions. There is a wealth of data and experimental studies regarding the activity of dapsone and related sulfones. In particular, there is a large amount of data regarding the bioavailability and pharmacokinetics of the drug.

It is also known in the prior art that dapsone has therapeutic activity against leprosy, dermatitis herpetiformis, actinomycotic mycetoma, asthma, malaria, rheumatoid arthritis, Kaposiís sarcoma, pneumocystis carinii (pneumonia), subcorneal pustular dermatosis and cystic acne, in patients in need of such therapy. However, since the acute or chronic toxicity of dapsone is unacceptable at the doses necessary to treat most diseases, it is not possible to use this compound for these indications in the presently available formulation(s).

In sharp contrast with modified-release formulation(s) of the present invention, sulfone(s) proposed to have anti-Alzheimers effect in humans have unfortunate adverse reactions including an increase of side effects, reduced efficacy of the drug and inevitably low or non-compliance.

At low instant doses, dapsone is known to cause anorexia, psychosis, agranulocytosis, peripheral neuritis, nausea, vomiting, headache, dizziness, tachycardia, nervousness, insomnia and skin disorders (in people with hypersensitivity). In most individuals given more than 150 mg daily, hemolysis and methemoglobinemia results. Due to these adverse reactions, its use as a therapy for the prevention of, or treatment of disease is severely limited in its current, orally-administered form (Jopling, 1983). Dapsone has a relatively long half-life in the plasma after rapid absorption from the proximal intestinal tract.

Drug efficacy generally depends upon the ability of the drug to reach its target in sufficient quantity to maintain therapeutic levels for the desired time period. The maximum intensity of the drug response occurs at the same time as the peak drug concentration in the blood. The drug concentration in the blood is in equilibrium with the drug at the receptor site, and the intensity of response is dependant upon the amount of drug at the receptor site.

Orally administered drugs must overcome several obstacles to reach their desired targets as compared to rectal administration in the form of modified-release suppositories. Before orally administered drugs enter the general circulation of the human body, they are absorbed into the capillaries of the upper gastrointestinal tract and are transported by the portal vein to the liver. The enzymatic activities, the pH found in gastrointestinal fluids or tissues, the concurrent intake of food and consequent agitation may inactivate the drug or cause the drug to dissolve poorly and consequently decrease compliance, increase the risk of side effects and substantially reduce the efficacy of the drug. In many cases, decreased bioavailability of orally administered drugs is a consequence of this “first pass” effect. In addition, following absorption in the intestine, orally administered drugs that are subjected to a “first pass” clearance by the liver maybe excreted into bile or converted into pharmacologically inactive or active metabolites.

### SUMMARY OF THE INVENTION

We have unexpectedly discovered that modified-release compositions of dapsone and other sulfone(s) will overcome limitations of the drug of the present invention for use as an effective therapeutic agent for treatment and prevention of certain diseases. Orally-administered modified-release dosage formulations of the present invention reduce toxicity, while maintaining a more stable plasma concentration. Modifed-release dosage forms of sulfone(s) of the present invention are characterized not only in that they convey the same or a larger amount of medicinal product than traditional problematic oral pharmaceutical preparations, but also retain compliance, reduced side effects and improved efficacy.

Modified-release dosage forms of sulfone(s) of the present invention including but not liming to subcutaneous gels, transdermal gels, solid, liquid or spray may be capable of releasing the active substance(s) at a constant rate (that is to say according to zero-order kinetics) up to complete release of the active substance thereby preventing the toxicity that is inherently found in instant-release dosage forms of sulfone(s) of the present invention. If absorption in any part of the intestinal tract might result in untoward effects, such absorption may be prevented, for example by enteric, acid-resistant, coat(s) or layer(s) where absorption from the stomach and proximal intestine is unwanted, as is the case with the sulfone(s) of the present invention.

Enteric coatings can be designed to remain intact in the acidic environment of the stomach protecting either the stomach from the drug or the drug from this environment, but to dissolve in the more alkaline environment of the proximal and distal gastrointestinal tract or rectum as a suppository. Little or no release takes place in the acidic medium of the stomach. However, as the drug leaves the stomach and enters the gastrointestinal tract, it is subjected to the intestinal fluids of pH 5.5-6.8.

In addition, further improvements in the therapeutic index can be raised by the sulfone(s) of the present invention by increasing the dissolution profile (release rate) of the active drug from tablets passing to more distal regions of the intestinal tract, thus decreasing the toxicity of metabolites that are produced more readily in the proximal intestinal tract than in the distal intestinal tract. Further adjustments in a modified-release dosage form may result in the selective absorption of the sulfone(s) of the present invention, wherein compatible agents such as absorption enhancing surfactants or swelling agents(gels), are added to one or more modified-release formulation(s) to selectively improve absorption or adsorption in the distal intestinal tract where water content of the intestinal content is less than in proximal segments, also resulting in reduced exposure of the sulfone(s) to metabolic enzymes that produce toxic metabolites.

Formulations of the present invention will also result in improved absorption of sulfone(s) in the distal segments of the intestinal tract where the propensity for metabolic production of toxic metabolites is less. Modified-release dosage compositions may include good quality granules, enteric-coated tablet(s), capsules, pills, powders and quality compression pressed granulates, micronized particles, micro-encapsulations and micro-sponges, consisting of the active therapeutic ingredient of the present invention.

As the drug leaves the stomach and enters the gastrointestinal tract, it is subjected to the intestinal fluids of pH 5.5-6.8. At this pH the enteric coat(s) or layer(s) commences to expose the drug to the action of the intestinal pH which the solubility of active compounds of the present invention is fairly high and consequently results in high dissolution and hence higher absorption into the blood stream.

The dissolution (rate of release) of the drug is relatively linear (a function of the rate limiting diffusion process through the enteric-coating) and inversely proportional to the coat(s) or layer(s) thickness. The variation of enteric coatings used in the present invention allow the active therapeutic ingredient(s) of varying dose regimes to disseminate either in a sustained, controlled, or delayed action thereby increasing compliance, reducing side effects and improving efficacy of the active therapeutic ingredient(s). The modified-release composition(s) may provide a delayed release delivery of the active therapeutic ingredient(s). Preferably the orally administered modified-release dosage form of the present will be administered into a nearly empty stomach, thereby preventing “bounce back” and instigating the “housekeeper” wave in the stomach.

For the enteric-coated tablet enteric-coatings that may be used can consist of pH sensitive polymers, phthalates including but not limited to cellulose acetate phthalate and ethylycellulose. Typically drug leaches, as an example, from an inert porous plastic (methacrylate) matrix, the release is independent of gastrointestinal motility, pH and enzymes, it is however dependent on the drugs solubility. The polymers are carboxylated and interact very little with water at low pH, while at high pH, as found in the distal gastrointestinal tract, including but not limited to the ascending, transverse and descending colon, polymer(s) ionize causing swelling and/or dissolving of the active hydrophilic polymer(s).

Additionally it will be understood, however, that the choice of modified-release dosage forms of the present invention maybe adjusted accordingly for any particular patient as this will depend upon a variety of factors including the age, route of administration, diet, time of administration, body weight, sex, general health, rate of excretion, drug combination and the severity of the particular disease undergoing therapeutic and preventative treatment.

Dapsone known in the prior art has therapeutic activity against leprosy, dermatitis herpetiformis, actinomycotic mycetoma, asthma, malaria, rheumatoid arthritis, Kaposiís sarcoma, pneumocystis carinii (pneumonia), subcorneal pustular dermatosis and cystic acne, in patients in need of such therapy. It has now surprisingly been found that dapsone, its didextrose sulfonate derivative (glucosulfone), its analogs thereof, for example sulfoxone, sulfetrone, thiazolsulfone, acedapsone, and its metabolites thereof, for example monoacetyldapsone, N-hydroxymonoacetyldapsone, N-hydroxydapsone, are also useful for preventing and for treating various conditions involving memory loss such as Alzheimer's disease and related neurodegenerative disorders. However, since the acute or chronic toxicity of dapsone is unacceptable at the doses necessary to treat most diseases, it is not possible to use this compound for these indications in the presently available formulation.

It has now surprisingly been found that modified release preparations of dapsone are significantly less toxic than regular, instant-release preparations of the drugs. This is an unexpected finding since dapsone is long-acting (Zuidema et al., 1986), having an average biological half-life of 28 hours in humans (Goodman and Gilman, 1996). New, modified release preparations of this compound that are the subject of this invention have the sought-after therapeutic activity, while significantly reducing drug toxicity. They also have minimal or are devoid of the side effects of instant release, orally-administered dapsone, that includes such effects as anorexia, psychosis, agranulocytosis, peripheral neuritis, hemolysis, methemoglobinemia nausea, vomiting, headache, dizziness, tachycardia, nervousness, insomnia and skin disorders (see Coleman et al., 1996; Jollow et al., 1995; Reilly et al., 1999; Tingle et al., 1997).

### Terminology

The term “galenical” as used here is defined as the task of applying pharmaceutical technology (galenics) for the development of drug dosage forms for given drug substances.

The term “bounce back” as used herein refers to the process where the pyloric sphincter closes and the stomach squeezes—as an example—a tablet back into the stomach and will not allow the tablet to pass into the intestine.

The term “housekeeper” wave as used herein refers to the relaxation of the pyloric sphincter—as instigated by a near empty stomach—followed by a strong contraction of the bowel lasting about 5 to 15 minutes creating a wave sweeping whatever is in the stomach into the intestine. Most things small enough to be swallowed will be moved into the intestine by this “housekeeper” wave.

The term “modified-release” dosage form as used herein is defined as the dosage form, with time course and/or location of the drug release, chosen to accomplish therapeutic or convenience objectives not offered by conventional dosage forms i.e. effective release of the drug into the systemic circulation treating or preventing certain diseases in a human being while increasing compliance, reducing side effects and improving efficacy of the active therapeutic ingredient(s).

The terms “sustained-release” and “controlled release” are not to be considered as synonyms, sustained-release describes the release of a drug from a dosage over a period of time, whereas controlled release describes a system in which the rate of drug-release is more precisely controlled than that in sustained release form.

The term “carrying capacities” refers to the amount of drug that a direct compression binder can “carry” into a good tablet.

The term “capping” defines when air is trapped and under released pressure the air expands and literally pops off the top of the tablet. This is rarely seen with good granules.

In the context of the present invention they may be used separately or in combination with each other and in many cases are related to each other.

### DETAILED DESCRIPTION OF THE INVENTION

The present invention includes modified-release dosage formulations of the compounds 4,4′-diaminodiphenylsulfone, its didextrose sulfonate derivative (glucosulfone), their analogs thereof, including sulfoxone, sulfetrone, thiazolsulfone, acedapsone, and its metabolites thereof, including monoacetyldapsone, N-hydroxymonoacetyldapsone, N-hydroxydapsone, and pharmaceutically and therapeutically acceptable salts thereof Pharmaceutically and therapeutically acceptable salts of the active ingredients of the present invention include, but are not limited to hydrochloride derivatives, sulphate, phosphate, citrate, fumarate, methanesulphonate, acetate, tartarate, maleate, lactate, mandelate, salicylate, succcinate, methylsulphonic acid derivatives, and cynnamic acid derivatives. Pharmaceutically acceptable carriers, excipients or diluents of the present invention may include but are not limited to sprays, gels transdermal or subcutaneous, liquids and solids incorporating lactose, sucrose, glucose, wax, mannitol, phthalates, methacrylate, silicic, sodium citrate, 1,2-Benzenedicarboxylic acid, ethylycellulose, dicalcium phosphate acid, absorption enhancing agents may include kaolin, sodium glycocholate, sodium caprate, n-lauryl-β-D-maltopyranoside, microcrystalline cellulose, hydrophilic polymer and compression binders may also include, sucrose, starch, hydroxypropylmethyl cellulose, polyethylene glycol, microcrystalline cellulose, hydroxymethyl cellulose, polyvinylpyrrolidone, carboxymethyl cellulose, alginates, gelatin, and mixtures thereof, disintegrating agents such as calcium carbonate, sodium starch glycholate, corn starch, tapioca starch, alginic acid, certain silicates, and sodium carbonate, lubricants and anti-adherents such as stearates including calcium stearate, magnesium stearate, talc, sodium lauryl sulfate, sodium ricinoleate, sodium tetradecylsulphate, sodium dioctylsulphosuccinate, poloxamer, glycerylmono stearate, a polysorbate, sorbitan monolaurate or a lecithin, physiological saline. Active ingredients also addressed by the present invention include any and all enantiomers and diastereomers, and any combination thereof, of sulfone(s) contemplated herein.

The above compounds are synthesized according to conventional methodology known to those skilled in the art (e.g., Yuasa, 1997), and may be prepared as a composition through combination with one or more therapeutically or pharmaceutically acceptable carrier(s), diluent(s) or excipient(s).

The modified-release dosage formulations of the present invention are based on a clinico-pharmacological rationale such as increase compliance, reduced side effects and improved efficacy. The actual dosage—quantity administered at a time—and the frequency of administrations will depend on the potency and the pharmacokinetic properties of the drugs.

If a more potent compound, or a compound with longer duration of therapeutic activity is chosen. the dose and the dosing frequency may be adjusted accordingly. Modified-release dosage forms of the present invention for oral administration may include but are not limited to capsules, tablets, pills, powders, granules, compression pressed granulates, micro-encapsulations, micro-spheres in a polymer film coated compressed tablet normally reserved for the stomach only—in this particular instance may be used in a controlled-release formulation of the present invention in the distal intestinal tract. Modification of particle size of active substance including various degrees of micronization will also result in improved absorption of sulfone(s) in the distal segments of the intestinal tract.

In such solid forms of the present invention, the active and inert compound(s) may be mixed with varying “carrying capacities”—to achieve the desired effect with at least one inert pharmaceutically acceptable or slightly active carrier.

Excipient(s) including fillers or binders of a central core may encompass starches, lactose, sucrose, glucose, mannitol, silicic acid and mixtures thereof.

Effective absorption enhancing agent(s) may include those such as kaolin, sodium glycocholate, microcrystalline cellulose, sodium caprate, n-lauryl-β-D-maltopyranoside and mixtures thereof.

Hydrophilic polymer binder(s) including for example, hydroxymethylcellulose, polyethylene glycol, polyvinylpyrrolidone, hydroxypropylmethylcellulose carboxymethylcellulose, alginates, gelatin, microcrystalline cellulose, sucrose, and mixtures thereof.

Disintegrating agent(s) such as calcium carbonate, sodium starch glycholate, corn starch, tapioca starch, alginic acid, certain silicates, and sodium carbonate,

Lubricant(s) such as talc, calcium stearate, magnesium stearate, sodium lauryl sulfate, sodium ricinoleate, sodium tetradecylsulphate, sodium dioctylsulphosuccinate, poloxamer, glycerylmonostearate, a polysorbate, sorbitan monolaurate or a lecithin and mixtures thereof.

A preferred embodiment of an oral modified-release formulation is an enteric-coated compressed tablet consisting of the active therapeutic ingredient of the present invention. This formulation could provide a delayed release, sustained release or controlled release delivery of the active therapeutic ingredient(s). For the enteric-coated tablet enteric-coating(s) that may be used consist of pH sensitive polymers, typically the polymers are carboxylated and interact very little with water at low pH, while at high pH, as found in the distal gastrointestinal tract, including but not limited to the ascending, transverse and descending colon, polymers ionize causing swelling, or dissolving of the active hydrophilic polymer(s).

Coatings can therefore be designed to remain intact in the acidic environment of the stomach protecting either the stomach from the drug or the drug from this environment, but to dissolve in the more alkaline environment of the proximal and distal gastrointestinal tract. Examples of the coating(s) that may be used include ethylycellulose, wax and cellulose acetate phthalate. Little or no release takes place in the acidic medium of the stomach. However, as the drug leaves the stomach and enters the gastrointestinal tract, it is subjected to the intestinal fluids of pH 5.5-6.8.

At this pH the enteric coat commences to expose the drug to the action of the intestinal pH which the solubility of active compounds of the present invention is fairly high, which then results in high dissolution and hence higher absorption into the blood stream. The rate of release (dissolution) of the drug is relatively linear (a function of the rate limiting diffusion process through the enteric-coating) and inversely proportional to the coating thickness. The variation of enteric coatings used in the present invention allow the active therapeutic ingredient(s) of varying dose regimes to disseminate either in a sustained, controlled, or delayed action thereby increasing compliance, reducing side effects and improving efficacy of the active therapeutic ingredient(s).

As will be apparent to those skilled in the art in the light of the foregoing disclosure, many alterations and modifications are possible in the practice of this invention without departing from the spirit or scope thereof.

### Biological Activities

The surprising effects of controlled release preparations of 4,4′-diaminodiphenylsulfone, its didextrose sulfonate derivative(s), its analogs and its metabolites, pharmaceutically and therapeutically acceptable salts can be demonstrated by the following tests:

1. Toxicological effects and pharmacological side effects of the compounds 4,4′-diaminodiphenylsulfone, glucosulfone, sulfoxone, sulfetrone, thiazolsulfone, acedapsone, monoacetyldapsone, N-hydroxymonoacetyldapsone and N-hydroxydapsone separately when administered orally in a conventional “instant release” formulation.

Groups of mice (males, 22-25 grams) are each administered orally one of the compounds 4,4′-diaminodiphenylsulfone, sulfoxone, sulfetrone, thiazolsulfone, glucosulfone, acedapsone, monoacetyldapsone, N-hydroxymonoacetyldapsone and N-hydroxydapsone in increasing concentrations, and the doses causing side effects are determined. Particular attention is paid to severe toxic manifestation such as the development of methemoglobinemia. Other known side effects of 4,4′-diaminodiphenylsulfone include anorexia, psychosis, agranulocytosis, peripheral neuritis, hemolysis, nausea, vomiting, dizziness, tachycardia, nervousness, insomnia and skin disorders, and the doses causing all such side effects are determined using statistical methodology.

Particular attention is paid to the possible development of nervousness, and specific test methods are used in order to define the dose levels of each compound that cause such an effect.

2. Toxicological effects and pharmacological side effects of the compounds 4,4′-diaminodiphenylsulfone, sulfoxone, sulfetrone, thiazolsulfone, glucosulfone, acedapsone, monoacetyldapsone, N-hydroxymonoacetyldapsone and N-hydroxydapsone when administered in a controlled release formulation.

Since clinically used “modified-release” preparations are designed for use in humans, they cannot be used in laboratory animals. It is therefore necessary to mimic the pharmacokinetics of the modified-release formulations of the drug in humans from a carefully selected modified-release preparation(s). Thus, in the present experiments the same dose as that given in the acute experiments described above is given as divided doses with 5 administered sub-doses, given at 2 hour intervals. Groups of animals treated this way are given increasing concentrations of the compound, and the doses causing side effects are determined. Particular attention is paid to severe toxic manifestation such as the development of methemoglobinemia. Expected side effects of 4,4′-diaminodiphenylsulfone include anticholinergic affects, and the doses causing such side effects are determined using statistical methodology. Particular attention is paid to the development of nervousness, and specific test methods are used in order to define the dose levels of 4,4′-diaminodiphenylsulfone that cause such side effects.

3. Pharmacological effects of the compounds 4,4′-diaminodiphenylsulfone, sulfoxone, sulfetrone, thiazolsulfone, glucosulfone, acedapsone, monoacetyldapsone, N-hydroxymonoacetyldapsone and N-hydroxydapsone when administered orally in a conventional “instant release” formulation.

Groups of mice (males, 22-25 grams) are administered orally one of the compounds 4,4′-diaminodiphenylsulfone, sulfoxone, sulfetrone, thiazolsulfone, glucosulfone, acedapsone, monoacetyldapsone, N-hydroxymonoacetyldapsone and N-hydroxydapsone in increasing concentrations and the doses causing anorexia, psychosis, agranulocytosis, peripheral neuritis, hemolysis, nausea, vomiting, dizziness, tachycardia, nervousness, insomnia or skin disorders are determined. Particular attention is paid to manifestation of nervousness. Other therapeutically important pharmacological effects of 4,4′-diaminodiphenylsulfone include methemoglobinemia, and the doses causing such effects are determined using pharmacological and statistical methodology.

4. Pharmacological effects of the compounds 4,4′-diaminodiphenylsulfone, sulfoxone, sulfetrone, thiazolsulfone, glucosulfone, acedapsone, monoacetyldapsone, N-hydroxymonoacetyldapsone and N-hydroxydapsone when administered in controlled release formulation.

Side effects of dapsone include anorexia, psychosis, agranulocytosis, peripheral neuritis, hemolysis, methemoglobinemia nausea, vomiting, dizziness, tachycardia, nervousness, insomnia and skin disorders, as well as other side effects that are described in the prior art.

The formation of the toxic dapsone metabolite, hydroxylaminedapsone, has now been found to be reduced, by certain controlled release formulations of dapsone, its didextrose sulfonate derivative(s), its analogs or metabolites. This application deals with controlled release formulations that avoid the exposure of said 4,4′-diaminodiphenylsulfone, its didextrose sulfonate derivative (glucosulfone), sulfoxone, sulfetrone, thiazolsulfone, acedapsone, monoacetyldapsone, N-hydroxymonoacetyldapsone and N-hydroxydapsone to the intestinal tract.

Various galenic formulations of 4,4′-diaminodiphenylsulfone, its didextrose sulfonate derivative (glucosulfone), sulfoxone, sulfetrone, thiazolsulfone, acedapsone, monoacetyldapsone, N-hydroxymonoacetyldapsone and N-hydroxydapsone may be used to avoid exposure of the drug of the present invention to the upper gastrointestinal tract. Numerous rectal and nasal delivery systems are known to those skilled in the art that allow the absorption of the drugs without exposure to the stomach or the upper intestines. Trans-dermal delivery systems that allow absorption of drugs through the skin have also been described in the prior art. Galenic formulations of 4,4′-diaminodiphenylsulfone, sulfoxone, sulfetrone, thiazolsulfone, glucosulfone, acedapsone, monoacetyldapsone, N-hydroxymonoacetyldapsone and N-hydroxydapsone may be dosed regularly (e.g., one to several times daily), intermittently (when needed), or as a combination of regular and intermittent dosages. When used regularly or intermittently, the galenic formulations of the present invention can be combined with each other, or with other dosage forms of the same drug, or with other drugs to be used by the patient.

Various enteric-coated tablets have also been described, or are being used therapeutically for unrelated pharmaceuticals. Enteric-coated tablets, pills, caplets etc. do not release the active components of the present invention into the stomach or upper intestines, but instead deliver the drug in the non-acid environment of the intestines after passing the stomach.

Modified-release dosage forms of the present invention include but are not limited to parenteral injection, nasal, transdermal, rectal administration or oral formulations except acute or instant-release formulations, including “delayed release” formulations (for example, see Roy et al., 1989), “sustained release” formulations (for example, see Yang and Swarbrick, 1986), “controlled release” formulations (for example, see U.S. Pat. Nos. 5,863,560 and 3,948,262), and also includes other oral formulations that are designed to offer therapeutic activity while avoiding toxicological effects and pharmacological side effects.

The pharmaceutical compositions of the present invention may be formulated for, oral administration in solid, liquid, spray or gel form, for parenteral injection, nasal, transdermal or rectal administration.

The compounds of the present invention may be administered by rectal suppositories such as those described in U.S. Pat. Nos. 4,368,185, 4,698,359 and 5,482,973, the disclosures of which are hereby incorporated by reference.

The compounds of the present invention may be administered by nasal delivery devices such as those described in U.S. Pat. Nos. 4,294,829, 4,624,965, 4,749,700, 5,250,287, 5,629,011 and 5,693,608, the disclosures of which are hereby incorporated by reference.

Preferably the compounds of the present invention may be administered by enteric-coated delivery devices such as those described in U.S. Pat. Nos. 4,704,295, 4,775,536 and 5,225,202, the disclosures of which are hereby incorporated by reference.

Preferably the compounds of the present invention may be administered by trans-dermal delivery devices such as those described in U.S. Pat. Nos.: 3,598,123; 4,292,302; 5,164,189; 5,312,627 and 5,464,387, the disclosures of which are hereby incorporated by reference.

The preferred selected dosage level chosen for the patient of the drug to be administered will be determined on an individual basis, and will be based on the pharmacological potency of the drug, age, route of administration, diet, time of administration, body weight, sex, general health, rate of excretion, drug combination, the condition, prior medical history of the patient being treated, and at least in part, on consideration of the individual's size, the symptoms, and the severity of the symptoms to be treated and the results sought. Also the carrying capacity of the drug may be adjusted to accommodate for the varying dosage regimes incorporated within the embodiment of the present invention. However, it is within the skill of the art to start doses of the compound at levels lower than required for to achieve the desired therapeutic and preventative effect and to gradually increase the dosage until the desired effect is achieved.

In general, preferred quantities of the compounds sufficient to eliminate an unwanted medical condition will be administered. The actual dosage (quantity administered at a time) and the frequency of administrations will depend on the potency and the pharmacokinetic properties of the drugs.

For example about from 5 mg to about 500 mg of a compound can be contained in one or more doses, one to ten such doses can be given daily. 1.1 ml is accepted as the maximum volume of solid a person can swallow—of course some people can swallow more and some less—and that means about 1.3 gm is the maximum acceptable tablet weight since the compressed tablet can have a density greater than 1.0. Most commercial products intended to be swallowed whole weigh 1.0 gm or less usually about 400-600 mg.

The amount of active ingredient of the present invention may be combined with carrier materials to produce one or more single dosage(s) form will depend on the patient and the particular mode of administration. For example, a modified-release formulation intended for oral administration of humans may contain from 5 mg to about 500 mg of active agent(s) compounded with an appropriate and convenient amount of carrier material(s) which may vary from about 5 to about 95 percent of the total composition.

Preferred dosage unit forms will generally contain between from about 5 mg to about 500 mg of active ingredient, typically 5, 10, 15, 20, 30 mg, 50 mg, 75 mg, 100 mg, 120 mg, 150 mg, 200 mg, 250 mg, 300 mg, 400 mg or 500 mg. However, it is within the skill of the art to start doses of the compound at levels lower than required for to achieve the desired therapeutic and preventative effect and to gradually increase the dosage until the desired effect is achieved.

One skilled in the art will recognize that modifications may be made in the present invention without deviating from the spirit or scope of the invention. The invention is illustrated further by the following examples which are not to be construed as limiting the invention or scope of the specific procedures described herein.

The examples which follow are intended to illustrate the invention.

### EXAMPLE 1

Preparation of controlled release tablets based on dapsone.

A granulate is prepared, according to the process described below, which is used for the preparation of one or more of the inner layers of the tablet. This granulate contains (per tablet):

| | |
|---|---|
| dapsone | 100 mg |
| mannitol | 10 mg |
| microcrystalline cellulose | 70 mg |
| sodium laurylsulphate | 5 mg |
| total tablet weight | 185 mg |
| carrying capacity | 40% dapsone 60% excipient (mostly com- |
| | pression binder) |

The manufacturing process consists in preparing a granulate by mixing together the amounts of substances as shown above and compressing into tablets. Tablets are coated with ethylycellulose.

### EXAMPLE 2

Dissolution Test

In order to evaluate the release properties of the complete tablets, the vane machine (described in USP XXIII) is used, working at 100 rpm and using as dissolution liquid a 0.01M HCl solution at 37 degrees Celcius. The release of the active substance is monitored by spectrophotometric determination using a sampling and automatic reading system.

A controlled release of the active substance is obtained in about 17 hours.

### EXAMPLE 3

Absorption Test

In order to evaluate the absorption of the sulfone from the distal intestinal tract with surfactant present in the tablet, tablets with and without surfactant are inserted into a distal intestinal pouch surgically created in a series of rats, with subsequent measurement of blood levels of dapsone. With surfactant present, absorption rate in the distal intestinal tract is greater.

### EXAMPLE 4

Coating Test

In order to evaluate the ability of a coating to protect the tablet from commencement of dissolution in the relatively acidic proximal intestinal tract, coated and non-coated tablets are placed in 0.01M HCl solution at 37 degrees Celcius. The release of the active substance is measured after 10 minutes by spectrophotometric determination. Then the respective tablets are placed in phosphate-buffered saline at pH 7.4 at the same temperature. The release of the active substance is again measured after 10 minutes by spectrophotometric determination. The smaller amount of dapsone release from coated tablets compared to un-coated tablets indicates that the coated tablets are resistant to dissolution in acid environment. No “capping”.

### EXAMPLE 5

Preparation of sustained release alginate tablets based on dapsone.

A compressed granulate is prepared, according to the process described below, which is used for the preparation of one or more of the inner layers of the tablet. This granulate contains (per tablet):

| | |
|---|---|
| dapsone | 100 mg |
| mannitol | 10 mg |
| microcrystalline cellulose | 50 mg |
| alginate | 15 mg |
| sodium laurylsulphate | 45 mg |
| total tablet weight | 230 mg |
| carrying capacity | 40% dapsone 60% excipient (mostly com- |
| | pression binder) |

The manufacturing process consists in preparing a granulate by mixing together the amounts of substances as shown above and compressing into tablets. Tablets are coated with alginate.

### EXAMPLE 6

Dissolution Test

In order to evaluate the release properties of the complete tablets, the vane machine (described in USP XXIII) is used, working at 100 rpm and using as dissolution liquid a 0.01M HCl solution at 37 degrees Celcius. The release of the active substance is monitored by spectrophotometric determination using a sampling and automatic reading system.

A sustained release of the active substance is obtained in about 22 hours.

### EXAMPLE 7

Absorption Test

In order to evaluate the absorption of the sulfone from the distal intestinal tract with surfactant present in the tablet, tablets with and without surfactant are inserted into a distal intestinal pouch surgically created in a series of rats, with subsequent measurement of blood levels of dapsone. With surfactant absent, absorption rate in the distal intestinal tract is greater.

### EXAMPLE 8

Coating Test

In order to evaluate the ability of a coating to protect the tablet from commencement of dissolution in the relatively acidic proximal intestinal tract, coated and non-coated tablets are placed in 0.01M HCl solution at 37 degrees Celcius. The release of the active substance is measured after 10 minutes by spectrophotometric determination. Then the respective tablets are placed in phosphate-buffered saline at pH 7.4 at the same temperature. The release of the active substance is again measured after 10 minutes by spectrophotometric determination. The smaller amount of dapsone release from coated tablets compared to un-coated tablets indicates that the coated tablets are resistant to dissolution in acid environment. No capping.

What is claimed is:

1. A method of treating or preventing in humans diseases selected from the group consisting of anti-inflammatory diseases, microbial diseases and diseases of dementia, said method comprising administering to said human a therapeutically effective amount of a modified-release dosage formulation of one or more therapeutic compositions selected from the group consisting of 4,4′-diaminodiphenyl-sulfone, a didextrose sulfonate derivative(s) of 4,4′-diaminodiphenylsulfone(glucosulfone), the analog acedapsone, the analog sulfoxone, the analog sulfetrone, the analog thiazolsulfone, the metabolite monoacetyldapsone, the metabolite N-hydroxymonoacetyldapsone, the metabolite N-hydroxydapsone, and therapeutically and pharmaceutically acceptable salts thereof, together with a pharmaceutically acceptable carrier, diluent or excipient.

2. A method according to claim 1, wherein the mode of administration of said therapeutic composition is selected from the group consisting of parenteral injection, nasal administration, transdermal administration, rectal administration and oral administration.

3. The method according to claim 1, wherein said modified-release dosage formulation is selected from the group consisting of sustained-release, delayed-release, controlled-release and combinations thereof

4. The method of claim 3, wherein the pH of said dosage formulation is about 5.5 to about 6.8.

5. The method according to claim 1 wherein said therapeutic composition is the didextrose sulfonate derivative of 4,4′-diaminodiphenylsulfone (glucosulfone.

6. The method according to claim 1 wherein said therapeutic composition is selected from the group consisting of monoacetyldapsone, N-hydroxymonoacetyldapsone and N—.

7. The method according to claim 1 wherein said therapeutic composition is selected from the group consisting of acedapsone, sulfoxone, sulfetrone and thiazolsulfone.

8. The method according to claim 1, wherein said therapeutic composition is administered while avoiding a first pass effect, first pass clearance, toxicological and pharmacological side effects.

9. The method according to claim 8 wherein said therapeutic composition is administered in the form of enteric coatings that are pH responsive polymers, ethylycellulose and celluloseacetate phthalates.

10. The method according to claim 1, wherein said diseases are selected from the group consisting of dementia, Leprosy, actinomycotic mycetoma, asthma, malaria, rheumatoid arthritis and kaposiis sarcoma.

11. The method according to claim 1 wherein said disease Alzheimers disease.

12. The method according to claim 1 wherein said disease is asthma.

13. The method according to claim 1 wherein said disease is selected the group consisting of dermatitis herpetiformis, pneumocytstis carinii, subcorneal pustular dermatosis and cystic acne.

14. The method according to claim 1 wherein said therapeutic composition is administered in a dosage of from about 5 mg to about 500 mg of active ingredient one to ten times daily.

15. A pharmaceutical composition for the treatment or prevention in humans diseases selected from the group consisting of anti-inflammatory diseases, microbial diseases and diseases of dementia, said composition comprising a therapeutically effective amount of a modified-release dosage formulation of one or more compounds selected from the group consisting of 4,4′-diaminodiphenyl-sulfone, a didextrose sulfonate derivative(s) of 4,4′-diaminodiphenylsulfone(glucosulfone), the analog acedapsone, the analog sulfoxone, the analog sulfetrone, the analog thiazolsulfone, the metabolite monoacetyldapsone, the metabolite N-hydroxymonoacetyldapsone, the metabolite N-hydroxydapsone, and therapeutically and pharmaceutically acceptable salts thereof, together with a pharmaceutically acceptable carrier, diluent or excipient.

16. The pharmaceutical composition of claim 15, wherein said modified-release dosage formulation is selected from the group consisting of sustained-release, delayed-release, controlled-release and combinations thereof.

17. The pharmaceutical composition of claim 15, wherein said therapeutic composition is in the form of enteric coatings that are pH responsive polymers, ethylycellulose and celluloseacetate phthalates.
